(19) United States
(12) Patent Application Publication
(10) Pub. No.: US 2025/0043311 A1
(43) Pub. Date: Feb. 6, 2025

(54) MICROSPHERE FACILITATED INTRACELLULAR DELIVERY AND ASSOCIATED METHODS AND DEVICES

(71) Applicant: VISICELL MEDICAL INC., San Diego, CA (US)

(72) Inventors: Mark L. STOLOWITZ, San Diego, CA (US); Mya S. THU, San Diego, CA (US); Johnny C. AKERS, San Diego, CA (US)

(73) Assignee: VISICELL MEDICAL INC., San Diego, CA (US)

(21) Appl. No.: 18/592,983

(22) Filed: Mar. 1, 2024

Related U.S. Application Data
(63) Continuation of application No. PCT/US2022/075870, filed on Sep. 1, 2022.
(60) Provisional application No. 63/239,640, filed on Sep. 1, 2021.

(51) Int. Cl. C12N 15/87; C12M 1/00; C12M 1/12

(52) U.S. Cl. CPC C12N 15/87; C12M 25/16; C12M 29/14

(57) ABSTRACT

Provided herein are methods and devices for microsphere facilitated intracellular delivery of exogenous cargo. In certain embodiments, the methods may comprise passing a solution comprising a plurality of cells and one or more exogeneous cargo through a matrix comprising a plurality of microspheres, wherein the one or more exogenous cargo is delivered to the plurality of cells. In certain embodiments, the methods may further comprise incubating the solution for a period of time after passing the solution through the matrix.

### PRIORITY CLAIM

The present application is a continuation of International Application No. PCT/US2022/075870, filed Sep. 1, 2022, which claims priority to U.S. Provisional Application No. 63/239,640, filed Sep. 1, 2021, the contents of which are hereby incorporated by reference in their entirety.

### BACKGROUND

Intracellular delivery of exogenous cargos is an indispensable process for studies involving modification of cells for numerous applications including diagnostic labeling, genome editing, cell-based therapy and biomanufacturing. However, conventional intracellular delivery systems, including electroporation, liposomes, particle bombardment, and microinjection, for loading exogeneous cargos into cells, suffer from significant shortcomings including low cell viability, cytotoxicity and inconsistent cargo delivery. A critical unmet need exists for improved approaches to intracellular delivery that is easily accessible to the end users, while equally adaptable to and scalable in a biomanufacturing setting for cost-effective manufacturing of modified cell products with high viability and retained functionality.

### SUMMARY

The present disclosure provides methods and devices for microsphere facilitated delivery of exogenous cargo into cells. As described herein, voids formed between microspheres provide sites for cell constriction to effect mechanoporation by cell squeezing, which allows for temporarily altering the porosity of the cell membrane to enable the introduction of exogenous cargos.

In some aspects, provided herein are methods for intracellular delivery comprising passing a solution comprising a plurality of cells and one or more exogeneous cargo through a matrix comprising a plurality of microspheres, wherein the one or more exogenous cargo is delivered to the plurality of cells. In certain embodiments, the method further comprises incubating the solution for a period of time after passing the solution through the matrix.

In some aspects, provided herein are methods for intracellular delivery comprising preparing a total volume of a solution in a column, the solution comprising a plurality of cells and one or more exogeneous cargo, the column comprising a matrix comprising a plurality of microspheres, wherein the total volume of the solution comprises a volume of the solution above the matrix and a volume of solution within the matrix; passing the total volume of solution through the matrix, wherein the one or more exogenous cargo is delivered to the plurality of cells, and wherein the volume of the solution above the matrix allows the plurality of cells to pass through the matrix before the total volume of the solution passes through the matrix. In certain embodiments, the methods further comprise incubating the solution for a period of time after passing the solution through the matrix.

In certain embodiments, the microspheres have a diameter of from about 3 micrometers (μm) to about 70 μm. In certain embodiments, the microspheres have a monodispersity of from about 1% to about 10%.

In certain embodiments, the microspheres have a diameter preferably selected from, but not limited to, from about 3 μm to about 10 μm, from about 20 μm to about 27 μm, from about 27 μm to about 32 μm, from about 27 μm to about 45 μm, from about 32 μm to about 38 μm, and from about 38 μm to about 45 μm.

In certain embodiments, a height of the matrix comprising the plurality of microspheres is from about 0.2 millimeters (mm) to about 2.0 mm.

In certain embodiments, the matrix comprises hexagonal close-packed lattices, cubic close-packed lattices, or hexagonal close-packed lattices and cubic close-packed lattices. In certain embodiments, a total volume of the matrix comprises from about 64% to about 74% of microspheres.

In certain embodiments, the microspheres are rigid. In certain embodiments, the microspheres are nonporous.

In certain embodiments, the microspheres comprise, consist of, or consist essentially of one or more materials selected from the group consisting of metal, silica, alumina, titania, zirconia, glass, ceramic, and organic polymer. In certain embodiments, the organic polymer is one or more selected from the group consisting of glycidyl methacrylate, polycaprolactone, polyetheretherketone, high density polyethylene, poly(methyl methacrylate), poly(lactic-co-glycolic acid), poly(L-lactic-co-hydroxymethyl glycolic acid), and polystyrene.

In certain embodiments, the period of time is from about 1 minute to about 60 minutes. In certain embodiments, the period of time is about 1 minute to about 30 minutes.

In certain embodiments, passing the solution occurs via centrifugal force. In certain embodiments, the centrifugal force is a speed of from about 100×g to about 2000×g. In certain embodiments, the centrifugal force is applied for about 3 minutes to about 30 minutes.

In certain embodiments, passing the solution occurs via pressure selected from the group consisting of pressure from a high-pressure gas source, pressure from a liquid pump, pressure from a liquid driven reservoir, and pressure from the application of a vacuum to the outlet.

In certain embodiments, the one or more exogenous cargo is selected from the group consisting of a therapeutic molecule, a gene editing tool, a reprogramming factor, a genetic modification tool, and an intracellular sensor, and an intracellular device. In certain embodiments, the gene editing tool may include, without limitation, one or more selected from the group consisting of guide RNAs (gRNAs), nucleases (e.g., RNA-guided nucleases, such as any CRISPR associated (Cas) protein, e.g., Cas9), ribonucleoproteins (RNP) (e.g., comprised of gRNA and RNA-guided nucleases), DNA templates, genome editing complexes, and vectors (e.g., plasmid DNA).

In certain embodiments, the cells comprise one or more selected from the group consisting of cells of the reproductive system, leukocyte cells, cells of the hematopoietic system, stromal cells, cells of the skeleton and musculature, cells of the neural system, cells of the digestive tract, cells of the skin, cells of the pituitary and hypothalamus, and cells of the adrenals and other endocrine glands. In certain embodiments, the cells comprise disease related cells.

In certain embodiments, the matrix is in a column. In certain embodiments, the column has an inlet and an outlet. In certain embodiments, the matrix is above the outlet. In certain embodiments, an internal diameter of the column is from about 5 mm to about 25 mm.

In certain embodiments, the matrix is supported by a polymeric frit or membrane filter. In certain embodiments, the polymeric frit comprises a material selected from the group consisting of polyamide, polyethylene, high density polyethylene, polytetrafluoroethylene, polyvinylidene difluoride, and Nylon 6. In certain embodiments, the membrane filter comprises a material selected from the group consisting of polytetrafluoroethylene, polyvinylidene difluoride, polypropylene, polyethersulfone, and polycarbonate.

In certain embodiments, the microspheres are comprised of sintered microspheres. In certain embodiments, the matrix comprises a frit.

In some aspects, devices are provided for intracellular delivery comprising a column and a matrix comprising a plurality of microspheres in the column.

In certain embodiments, the column has an inlet and an outlet. In certain embodiments, the matrix is positioned above the outlet.

In certain embodiments, the microspheres have a diameter of from about 3 μm to about 70 μm. In certain embodiments, the microspheres have a monodispersity of from about 1% to about 10%.

In certain embodiments, the microspheres have ranges of diameters, wherein the ranges are preferably selected from, but not limited to, one of from about 3 μm to about 10 μm, from about 20 μm to about 27 μm, from about 27 μm to about 32 μm, of from about 27 μm to about 45 μm, of from about 32 μm to about 38 μm, and of from about 38 μm to about 45 μm.

In certain embodiments, a height of the matrix is from about 0.2 mm to about 2.0 mm.

In certain embodiments, the matrix comprises hexagonal close-packed lattices, cubic close-packed lattices, or hexagonal close-packed lattices, and cubic close-packed lattices. In certain embodiments, a total volume of the matrix comprises from about 64% to about 74% of microspheres.

In certain embodiments, the microspheres are rigid. In certain embodiments, the microspheres are nonporous.

In certain embodiments, the microspheres comprise, consist of, or consist essentially of, one or more materials selected from the group consisting of metal, silica, alumina, titania, zirconia, glass, ceramic, and organic polymer. In certain embodiments, the organic polymer is one or more selected from the group consisting of glycidyl methacrylate, polycaprolactone, polyetheretherketone, high density polyethylene, poly(methyl methacrylate), poly(lactic-co-glycolic acid), poly(L-lactic-co-hydroxymethyl glycolic acid), and polystyrene.

In certain embodiments, an internal diameter of the column is about 5 mm to about 25 mm.

In certain embodiments, the matrix is supported by a polymeric frit or membrane filter. In certain embodiments, the polymeric frit comprises a material selected from the group consisting of polyamide, polyethylene, high density polyethylene, polytetrafluoroethylene, polyvinylidene difluoride, and Nylon 6. In certain embodiments, the membrane filter comprises a material selected from the group consisting of polytetrafluoroethylene, polyvinylidene difluoride, polypropylene, polyethersulfone, and polycarbonate.

In certain embodiments, the microspheres are comprised of sintered microspheres. In certain embodiments, the matrix comprises a frit.

In some embodiments, the microspheres reside within a cylindrical column having a flange at the inlet (top) and the bed of microspheres above the outlet (bottom), wherein the external diameter of the column fits within a liquid handling receiver designed to be processed in a centrifuge, wherein the flange at the top of the column supports the column above the bottom of liquid handling receiver designed, wherein the internal diameter of the column is from about 5 mm to about 25 mm, wherein the height of the bed of microspheres is from about 0.2 mm to about 2.0 mm, wherein a plurality of cells in the presence of exogeneous cargos is propelled through the bed of microspheres by centrifugal force, and wherein the intracellular loading of exogenous materials into the target cells in suspension is introduced when the device is subjected to the centrifugal force.

In some embodiments, the microspheres reside within a cylindrical column having tubing connectors at an inlet and an outlet, wherein the internal diameter of the column is from about 5 mm to about 25 mm, wherein the height of the bed of microspheres is from about 0.2 mm to about 2.0 mm, and wherein a plurality of cells in the presence of exogeneous cargos is propelled through the bed of microspheres by one of either a liquid pump, pressure driven liquid reservoir and application of vacuum to the outlet of the column.

In some embodiments, the bed of microspheres in the cylindrical column is supported by either a polymeric frit or membrane filter placed either below the bed or both above and below the bed, wherein the polymeric frit or membrane filter retains the microspheres but permits the unrestricted passage of cells and exogenous cargos, and wherein the frit or membrane can be further supported by a rigid metallic screen or porous polymeric disc.

In some embodiments, the bed of microspheres can be comprised of sintered microspheres prepared by sintering compressed microspheres at elevated temperature to produce a rigid bed of fused microspheres, wherein the bed is comprised of polymeric microspheres having either a diameter of from about 10 microns to about 60 microns, or a range of diameters of from about 3 microns to about 45 microns, wherein the microspheres are organized into one of hexagonal close-packed, cubic close-packed and a combination of hexagonal and cubic close packed structures, and wherein the volume of the microspheres occupy from about 64% to about 74% of the total bed volume. In certain embodiments, the microspheres have a narrow size distribution or monodispersity of from about 1% to about 10%.

### BRIEF DESCRIPTION OF THE DRAWINGS

FIG. 1 depicts a method of intracellular delivery in accordance with one or more embodiments of the present disclosure.

FIG. 2 depicts the appearance of a close-packed plane of microspheres comprised of spheres and triangular voids that is encountered by cells and exogeneous cargo while performing the method of intracellular delivery in accordance with one or more embodiments of the present disclosure.

FIG. 3 depicts the movement of cells and exogenous cargo through a bed of microspheres during centrifugation using a swinging bucket rotor.

FIGS. 4a-4d show various embodiments of columns of the present disclosure. FIG. 4a shows a cross-sectional view of a cylindrical column for intracellular delivery having a flange at the inlet and a bed of microspheres above the outlet in accordance with one or more embodiments of the present disclosure. FIG. 4b shows a cross-sectional view of a cylindrical column for intracellular delivery having a flange at the inlet and a bed of microspheres above the outlet, wherein a bed of microspheres is placed between two filter membranes and supported by a metallic screen or porous polymeric disc above the outlet in accordance with one or more embodiments of the present disclosure. FIG. 4c shows cross-sectional views of cylindrical columns for intracellular delivery having a flange at the inlet and a bed of sintered microspheres above the outlet in accordance with one or more embodiments of the present disclosure. In the “spin column” configuration (left), the outlet is depicted as a male luer adapter, whereas in the “spin cup” configuration (right), the outlet has no connector associated with it. FIG. 4d shows cross-sectional views of cylindrical columns for intracellular delivery having a flange at the inlet and a bed of sintered microspheres above the outlet in accordance with one or more embodiments of the present disclosure. The columns differ with respect to their diameter but require that the height of the solution containing the cells and exogenous cargos must be sufficient to enable the cells to transverse the bed of microspheres before the solution is depleted.

FIGS. 5a-5b show the geometry and derivation of equations related to triangular voids. FIG. 5a depicts the geometry of the triangular void associated with close-packed structures involving equal spheres. FIG. 5b summarizes the derivation of equations to determine the radius of a sphere inscribed within the triangular void and to estimate the approximate area of the triangular void.

FIG. 6 shows a graphical representation of the relationship between the diameter of the microsphere and the approximate area of the corresponding triangular void.

FIG. 7 depicts diagonal close-packed and cubic close-packed structures involving equal spheres.

FIGS. 8a-8b depict 3-dimensional arrangements of voids. FIG. 8a depicts the 3-dimensional arrangement of identical spheres in tetrahedral voids. FIG. 8b depicts the 3-dimensional arrangement of identical spheres in octahedral voids.

FIG. 9 summaries the equations used to determine the volumes of tetrahedral and octahedral voids as a function of the radii of spheres inscribed within the voids.

FIG. 10 shows a graphical representation of the relationship between the diameter of the microsphere (μm) and the volumes (μm³) of the corresponding tetrahedral (black circle) and octahedral (black diamond) voids. The approximate volumes of therapeutic and regenerative cells with respect to cell squeezing are also shown: T-cell volume (268 μm³), natural killer (NK) cell volume (382 μm³), neural stem cell (NSC) volume (1767 μm³), mesenchymal stem cell (MSC) volume (3054 μm³).

FIG. 11 shows microscope images of peripheral blood-derived T-cells modified with VMI-Trac Ultra nanoparticles and stained with Prussian Blue. The images are from an experiment performed with a PEEK frit with average porosity of 10 μm. The panel on the left (“Unlabeled”) shows cells processed in parallel in the absence of exogenous cargo (i.e., VMI-Trac Ultra nanoparticles). The panel on the right (“Labeled”) shows cells stained with Prussian Blue, which is used to detect iron, to visualize the presence of iron-based VMI-Trac Ultra nanoparticles.

FIGS. 12a-12d show results from intracellular delivery of VMI-Trac Ultra nanoparticles into peripheral blood-derived T-cells. FIG. 12a shows microscope images of T-cells modified with VMI-Trac Ultra nanoparticles and stained for 1 hour with Prussian Blue to visualize the presence of iron-based VMI-Trac Ultra nanoparticles. Images are from experiments performed with PEEK frits with average porosities of 10 μm, 5 μm, or 2 μm. The scale bar represents 25 μm. Arrows indicate examples of intracellular delivery of VMI-Trac Ultra nanoparticles visualized by Prussian Blue staining. Arrowheads indicate extracellular VMI-Trac Ultra nanoparticle clusters visualized by Prussian Blue staining. FIG. 12b shows microscope images of T-cells modified with VMI-Trac Ultra nanoparticles and stained for 1 hour with Prussian Blue and/or labeled with the fluorescent VMI-Trac Duo reagent, which causes VMI-Trac Ultra nanoparticles to fluoresce. The images are from an experiment performed with a PEEK frit with average porosity of 10 μm. The left panels show T-cells stained for 1 hour with Prussian Blue to visualize the presence of iron-based VMI-Trac Ultra nanoparticles. The middle panels show fluorescence of VMI-Trac Ultra nanoparticles to visualize the presence of VMI-Trac Ultra nanoparticles labeled with the fluorescent VMI-Trac Duo reagent. The right panels are an alignment of the images from the left and middle panels showing that the fluorescent signal co-localized with Prussian Blue confirming the presence of VMI-Trac Ultra nanoparticles. FIG. 12c shows the total cell recovery of T-cells processed with the spin-column fitted with different porosity frits (10 μm, 5 μm, or 2 μm) and determined by trypan blue staining. Recovery of T-cells was determined by counting the number (%) of cells in the sample versus the number (%) of cells recovered. “Control” represents cells that were not processed through the frits. The mean and standard error of measurement (SEM) of triplicate experiments were plotted. FIG. 12d shows the viability of T-cells processed with the spin-column fitted with different porosity frits and determined by trypan blue staining. Viability reflects the ratio of live/dead cells. “Control” represents cells that were not processed through the frits. The mean and SEM of triplicate experiments were plotted.

FIG. 13 provides an example of a schematic of a die used to prepare sintered microsphere frits in an embodiment herein. The die may be used to compress microspheres prior to sintering at an elevated temperature or to simply compress microspheres prior to transferring them to spin columns whereby eliminating the need for slurry packing. The die may be used in conjunction with a benchtop press with a rating of from about 5 to about 15 tons.

### DETAILED DESCRIPTION

Provided herein are methods and devices for microsphere facilitated intracellular delivery, which offer significant advantages over prior art approaches with respect to simplicity, throughput, and ease of operation. Microsphere facilitated intracellular delivery allows for mechanoporation of cells, which is a method for intracellular delivery in which mechanical energy is used to create transient membrane pores in cells. Mechanoporation by “cell squeezing” through the voids formed between packed microspheres results in temporarily altering the porosity of the cell membrane to enable the introduction of exogeneous cargos.

After reading this description it will become apparent to one skilled in the art how to implement the invention in various alternative embodiments and alternative applications. However, all the various embodiments of the present disclosure will not be described herein. It will be understood that the embodiments presented here are presented by way of example only, and not limitation. As such, this detailed description of various alternative embodiments should not be construed to limit the scope or breadth of the present disclosure as set forth below.

The detailed description is divided into various sections only for the reader's convenience and disclosure found in any section may be combined with that in another section. Titles or subtitles may be used in the specification for the convenience of a reader, which are not intended to influence the scope of the present disclosure.

Unless the context indicates otherwise, it is specifically intended that the various features of the invention described herein can be used in any combination.

### Definitions

All numerical designations are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1% of the specified amount.

### Microspheres

Aspects of the present disclosure relate to microspheres.

In certain embodiments, the microspheres may be rigid. In certain embodiments, the microspheres may be non-porous.

In certain embodiments, the microspheres may have a diameter of from about 3 micrometers (μm) to about 70 μm. In certain embodiments, the microspheres may have a diameter of from about 3 μm to about 60 μm. In certain embodiments, the microspheres may have a diameter of from about 3 μm to about 20 μm. In certain embodiments, the microspheres may have a diameter of from about 6 μm to about 18 μm.

In certain embodiments, the microspheres may have a size distribution, otherwise referred to as monodispersity, which refers to the variation of the microspheres in diameter, of from about 1% to about 10%. In certain embodiments, the microspheres may have a monodispersity of about 1%, about 2%, about 3%, about 4%, about 5%, about 6%, about 7%, about 8%, about 9%, or about 10%. In certain embodiments, the microspheres may have a monodispersity of 1-3%, 4-6%, 5-8%, 7-8%, or 9-10%.

In certain embodiments, the microspheres have ranges of diameters, wherein the ranges are preferably selected from, but not limited to, one of from about 3 μm to about 10 μm, from about 20 μm to about 27 μm, from about 27 μm to about 32 μm, of from about 27 μm to about 45 μm, of from about 32 μm to about 38 μm, and of from about 38 μm to about 45 μm.

In certain embodiments, the ranges of microspheres are obtained by sequential sieving using American Standard Test Sieve Series (ASTM).

In certain embodiments, the microspheres may be provided in a bed (also referred to as a matrix). In certain embodiments, a height of the bed (i.e., matrix) of microspheres may be from about 0.2 millimeters (mm) to about 5.0 mm. In certain embodiments, a height of the bed (i.e., matrix) of microspheres may be from about 0.2 mm to about 1.0 mm, from about 0.5 mm to about 1.0 mm, from about 1.0 mm to about 1.5 mm, from about 1.5 mm to about 2.0 mm, from about 2.0 mm to about 2.5 mm, from about 2.5 mm to about 3.0 mm, from about 3.0 mm to about 3.5 mm, from about 3.5 mm to about 4.0 mm, from about 4.0 mm to about 4.5 mm, from about 4.5 mm to about 5.0 mm.

In certain embodiments, the microspheres may pack together to form spaces or voids between the microspheres. In certain embodiments, the voids may provide for cell constriction, cell recovery, or a combination thereof.

In certain embodiments, the bed (i.e., matrix) of microspheres may comprise hexagonal close-packed lattices. In certain embodiments, the microspheres may comprise cubic close-packed lattices. In certain embodiments, the microspheres may comprise hexagonal close-packed lattices and cubic close-packed lattices.

In certain embodiments, the microspheres may comprise from about 64% to about 74% of the total bed volume of the bed (i.e., matrix) of microspheres. In certain embodiments, the microspheres may comprise about 64%, about 65%, about 66%, about 67%, about 68%, about 69%, about 70%, about 71%, about 72%, about 73%, about 74% of the total bed volume of the bed (i.e., matrix) of microspheres. In certain embodiments, the microspheres may comprise at least 64%, at least 65%, at least 66%, at least 67%, at least 68%, at least 69%, at least 70%, at least 71%, at least 72%, at least 73%, at least 74% of the total bed volume of the bed (i.e., matrix) of microspheres. In certain embodiments, the microspheres may comprise no more than 64%, no more than 65%, no more than 66%, no more than 67%, no more than 68%, no more than 69%, no more than 70%, no more than 71%, no more than 72%, no more than 73%, no more than 74% of the total bed volume of the bed (i.e., matrix) of microspheres.

In certain embodiments, the microspheres may be polymeric microspheres preferably selected from, but not limited to, high density polyethylene (HDPE), polyetheretherketone (PEEK), poly(methyl methacrylate) (PMMA) and polystyrene (PS) microspheres.

In certain embodiments, the microspheres may include sintered polymeric microspheres. For example, the sintered polymeric microspheres may be prepared by sintering a compressed bed of microspheres at an elevated temperature to produce a rigid bed of fused microspheres. In certain embodiments, the rigid bed of fused microspheres may be a frit. In certain embodiments, the rigid bed of fused microspheres may be comprised of polymeric microspheres preferably selected from, but not limited to, high density polyethylene (HDPE), polyetheretherketone (PEEK), poly(methyl methacrylate) (PMMA) and polystyrene (PS).

In certain embodiments, the microspheres may not have any surface modifications. In certain embodiments, the microspheres may have one or more surface modifications. For example, in certain embodiments, the microspheres may have a surface modification to minimize their potential for ionic and nonspecific adsorption of cells and exogenous cargos. Methods for surface modification of the microspheres are well documented and will be familiar to those with skill in the art of biocompatible surface chemistries.

### Cells

Cells suitable for use in conjunction with the methods and devices disclosed herein include, but are not limited to, cells of the reproductive system, e.g. oocytes, spermatozoa, leydig cells, embryonic stem cells, amniocytes, blastocysts, morulas, and zygotes; leukocytes, e.g. peripheral blood leukocytes, spleen leukocytes, lymph node leukocytes, hybridoma cells, T cells (cytotoxic/suppressor, helper, memory, naive, and primed), B cells (memory and naive), monocytes, macrophages, granulocytes (basophils, eosinophils, and neutrophils), natural killer cells, natural suppressor cells, thymocytes, and dendritic cells; cells of the hematopoietic system, e.g. hematopoietic stem cells (CD34+), proerythroblasts, normoblasts, promyelocytes, reticulocytes, erythrocytes, pre-erythrocytes, myeloblasts, erythroblasts, megakaryocytes, B cell progenitors, T cell progenitors, thymocytes, macrophages, mast cells, and thrombocytes; stromal cells, e.g. adipocytes, fibroblasts, adventitial reticular cells, endothelial cells, undifferentiated mesenchymal cells, epithelial cells including squamous, limbal cells, cuboid, columnar, squamous keratinized, and squamous non-keratinized cells, and pericytes; cells of the skeleton and musculature, e.g. myocytes (heart, striated, and smooth), osteoblasts, osteoclasts, osteocytes, synoviocytes, chondroblasts, chondrocytes, endochondral fibroblasts, and perichondrial fibroblasts; cells of the neural system, e.g. astrocytes (protoplasmic and fibrous), microglia, oligodendrocytes, and neurons; cells of the digestive tract, e.g. parietal, zymogenic, argentaflin cells of the duodenum, polypeptide-producing endocrine cells (APUD), islets of langerhans (alpha, beta, and delta), hepatocytes, and Kupffer cells; cells of the skin, e.g. keratinocytes, langerhans, and melanocytes; cells of the pituitary and hypothalamus, e.g. somatotropic, mammotropic, gonadotropic, thyrotropic, corticotropin, and melanotropic cells; cells of the adrenals and other endocrine glands, e.g. thyroid cells (C cells and epithelial cells); adrenal cells; and tumor cells.

Additional disease-related cells suitable for use in conjunction with the methods and devices disclosed herein include, but are not limited to, Burkitt lymphoma cells, choriocarcinoma cells, adenocarcinoma cells, non-Hodgkin's B and T cell lymphoma cells, fibrosarcoma cells, neuroblastoma cells, plasmacytoma cells, rhabdomyosarcoma cells, carcinoma cells of the pharynx, renal adenocarcinoma, hepatoma cells, fibrosarcoma cells, myeloma cells, osteosarcoma cells, teratoma cells, teratomal keratinocytes, lung carcinoma cells, colon adenocarcinoma cells, lung adenoma cells, renal carcinoma cells, rectum adenocarcinoma cells, chronic myelogenous leukemia cells, ileocecal adenocarcinoma cells, hairy cell leukemia cells, acute myelogenous leukemia cells, colon carcinoma cells, cecum carcinoma and adenocarcinoma cells, leukemiacecum adenocarcinoma cells, pancreatic carcinoma, Wilm's tumor cells, prostate adenocarcinoma cells, renal leiomyoblastoma cells, bladder carcinoma cells, plasmacytoma cells, teratocarcinoma cells, breast carcinoma, epidermoid carcinoma of the cervix, ovarian teratocarcinoma, myeloma cells, T and B cell lymphoma cells, amelanotic melanoma cells, cervical carcinoma cells, rhabdomyosarcoma, hepatoma, medullary Thyroid carcinoma cells, malignant melanoma cells, glioblastoma cells, plasma cell leukemia, endometrial adenocarcinoma, squamous cell carcinoma, pancreatic adenocarcinoma, astrocytoma, gastric adenocarcinoma, pulmonary mucoepidermoid carcinoma cells, myeloid leukemia cells, EBY-transformed B cells, renal cell adenocarcinoma, acute leukemia, B cell plasmacytoma, acute lymphocytic leukemia, cutaneous T lymphoma, T cell leukemia, acute lymphoblastic leukemia, HIV+ T cells, medulloblastoma, B cells from sickle cell disease, acute monocytic leukemia, adrenocortical carcinoma, Bowes Melanoma and hepatocellular carcinoma.

### Exogenous Cargo

In certain embodiments, exogenous cargo used with the methods and devices herein may include any exogenous material to be delivered to a cell.

For example, exogenous cargo suitable for use in conjunction with the methods and devices disclosed herein may include, but is not limited to, therapeutic molecules, gene editing tools, reprogramming factors, genetic modification tools and intracellular sensors and devices.

In certain embodiments, therapeutic molecules may include, but are not limited to, siRNA, mRNA, transcription factors and genome editing complexes.

In certain embodiments, gene editing tools may include, but are not limited to, guide RNAs (gRNAs), nucleases (e.g., RNA-guided nucleases, such as any CRISPR associated (Cas) protein, e.g., Cas9), ribonucleoproteins (RNP) (e.g., comprised of gRNA and RNA-guided nucleases), DNA templates, genome editing complexes, and vectors (e.g., plasmid DNA). In some embodiments, the gene editing tool may comprise a plasmid vector, for example, a plasmid comprising a CRISPR-Cas (e.g., CRISPR-Cas9) system. In some embodiments, the gene editing tool may be a genetic material for the expression of certain proteins or receptors, for example, Chimeric Antigen Receptor (CAR) for cells such as T Lymphocytes and Natural Killer cells.

In certain embodiments, reprogramming factors may include, but are not limited to, miRNA, small molecules, transcription factors and vectors (e.g., plasmid DNA).

In certain embodiments, genetic modulation tools include, but are not limited to, miRNA, mRNA, siRNA, vectors (e.g., plasmid DNA), DNA/RNA oligonucleotides and artificial chromosomes.

In certain embodiments, intracellular sensors and devices may include, but are not limited to, small molecules (for example, without limitation, drugs, detectable labels (e.g., fluorophores, metal chelates), siRNA, miRNA), peptides, proteins, antibodies, quantum dots, nanoparticles, molecular beacons, carbon nanotubes and nanodevices.

### Devices for Intracellular Delivery

Devices for microsphere facilitated intracellular delivery are provided herein.

In certain embodiments, devices for intracellular delivery may include a column. In certain embodiments, the column may be a cylindrical column. In certain embodiments, the column may be for use in a centrifuge. For example, in certain embodiments, the column may be a spin column. In certain embodiments, the spin column may include a male luer adapter. In certain embodiments, the column may be a spin cup. In certain embodiments, the spin cup may have no connector or adapter.

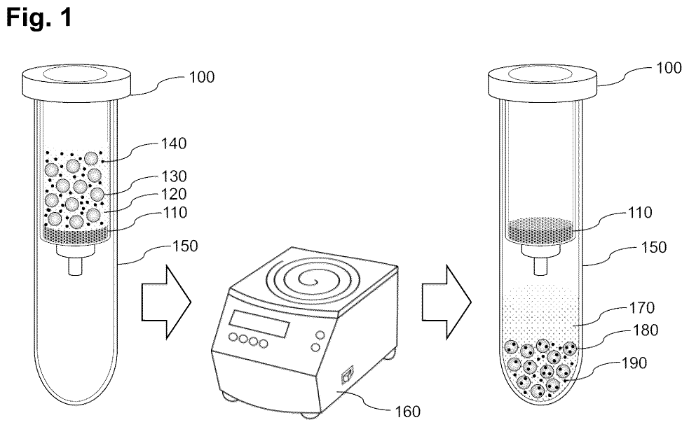

In certain embodiments, spin columns are comprised of an inner processing column that is placed in an outer receiving column. To prevent the inner column from slipping to the bottom of the outer column, a flange at the top of the inner column holds it in place above the bottom of the outer column (see, for example, FIG. 1). In certain embodiments, the external diameter of the column may fit within a liquid handling receiver designed to be processed in a centrifuge. In certain embodiments, the column includes a flange at the top of the column, which supports the column above the bottom of liquid handling receiver designed.

In certain embodiments, the internal diameter of the column may be from about 5 mm to about 25 mm. In certain embodiments, the internal diameter of the column may be from about 5 mm to about 10 mm, from about 10 mm to about 15 mm, from about 15 mm to about 20 mm, or from about 20 mm to about 25 mm. In certain embodiments, the internal diameter of the column may be about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, or about 25 mm.

In certain embodiments, the column has an inlet and an outlet. In certain embodiments, the column may comprise a bed (i.e., matrix) of microspheres. In certain embodiments, the bed (i.e., matrix) of microspheres is located above the outlet.

In certain embodiments, the bed (i.e., matrix) of microspheres may be supported by a polymeric frit or membrane filter. The frit or membrane may act as a physical support to hold the bed above the bottom of the column. The porosity of the frit or membrane retains the microspheres but permits the passage of cells. In certain embodiments, the polymeric frit or membrane filter may be placed below the bed, above the bed, or both above and below the bed. In certain embodiments, the polymeric frit may be prepared from polyamide, polyethylene, high density polyethylene, polytetrafluoroethylene, polyvinylidene difluoride, or Nylon 6. In certain embodiments, the membrane may be prepared from polytetrafluoroethylene, polyvinylidene difluoride, polypropylene, polyethersulfone, or polycarbonate.

In certain embodiments, the device may include one or more columns to allow for microsphere-based multiple or parallel mechanoporation of cells. For example, in certain embodiments, the device may include multiple columns that can be processed simultaneously in a centrifuge. In certain embodiments, the number of columns may be an amount that a centrifuge rotor will accommodate. In certain embodiments, the device may be a spin column plate comprised of parallel columns. In certain embodiments, the parallel columns may include 12, 24, 48, or 96 columns. In certain embodiments, the parallel columns may be in a 2×3 format in a centrifuge rotor with a microwell plate adapter.

### Methods of Intracellular Delivery

Aspects of the present disclosure relate to methods of intracellular delivery. In certain embodiments, the method of intracellular delivery includes a step of preparing a solution that includes cells and one or more exogenous cargo as described herein. In certain embodiments, the method of intracellular delivery includes a step of passing or flowing the solution including the cells and one or more exogenous cargo through a plurality of microspheres. In certain embodiments, the one or more endogenous cargo may be delivered to the cells. In certain embodiments, the plurality of microspheres may be a bed (i.e., matrix) of microspheres. In certain embodiments, the microspheres may be any of the microspheres described herein. In certain embodiments, the microspheres may pack together to form spaces or voids between the microspheres. In certain embodiments, the voids may provide for cell constriction, cell recovery, or a combination thereof.

As described in more detail herein, the passage of the cells through the voids between the microspheres may result in mechanoporation by cell squeezing (constriction), allowing for temporary alteration of the porosity of the cell membrane and delivery of exogenous cargo to the cell. In certain embodiments, passage of the cells into the voids may result in an alteration of the cell. In certain embodiments, the alteration may comprise altering the porosity of the cell membrane by creating pores in the cell membrane. In certain embodiments, altering the porosity of the cell membrane allows for exogenous cargo to be delivered to the cell. In certain embodiments, the alteration of the cell may comprise decreasing the volume of the cell. In certain embodiments, passage of cells through the voids allows for cells recovery, for example, for cells to recover their volume.

In certain embodiments, the solution comprising cells and exogenous cargo that is passed through the microspheres must be at a sufficient volume to enable the cells to traverse the bed of microspheres before the volume of the solution above the bed of microspheres is depleted. If the volume of the solution is insufficient to ensure that the cells clear the matrix, cells can become trapped, which clogs the matrix and dehydrates the cells. Thus, to ensure high recovery and high viability of cells, the process must be continuous until all cells are cleared from the matrix.

In certain embodiments, following the step of passing or flowing the solution through the plurality of microspheres, the method of intracellular delivery further includes a step of incubating the solution including the cells and one or more exogenous cargos. This incubation step provides the time required by the cells for the transient pores that formed in the membrane to close and for the volume to return to normal. In certain embodiments, the incubation step may occur for a period of time that allows the pores that formed in the cell membrane as a result of mechanoporation to close. In certain embodiments, the incubation step may occur for about 1 minute to about 120 minutes. In certain embodiments, the incubation step may occur for about 1 minute to about 60 minutes. In certain embodiments, the incubation step may occur for, without limitation, 1 minute, 3 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 75 minutes, 90 minutes, 120 minutes. For example, in certain embodiments, the incubation step may occur for 5 minutes. In certain embodiments, the incubation step may occur for 30 minutes.

In certain embodiments, the incubation step may occur at, for example, 4° C., room temperature, or 37° C.

### Some Embodiments of the Present Disclosure

FIG. 1 depicts a method of intracellular delivery in accordance with one or more embodiments of the present disclosure. A column designed to be processed in a centrifuge (e.g., a “spin column”) 100 with a bed of microspheres 110 supported above the outlet is loaded with a volume of solution 120 containing cells 130 and exogeneous cargos 140. The column is placed in a centrifuge tube 150 and the centrifuge tube is placed in a centrifuge 160. The tube is centrifuged for a period of from about 3 minutes to about 30 minutes at a speed of from about 100×g to about 2000×g. After centrifugation, the processed cells modified by incorporation of exogeneous cargos 180 are pelleted at the bottom of the centrifuge tube and excess exogeneous cargos 190 are found in either the supernatant 170, the pellet or both the supernatant and the pellet. The volume of solution 120 containing cells 130 and exogeneous cargos 140 must be sufficient to enable the cells to transverse the bed of microspheres 110 before the volume of solution above the bed of microspheres is depleted.

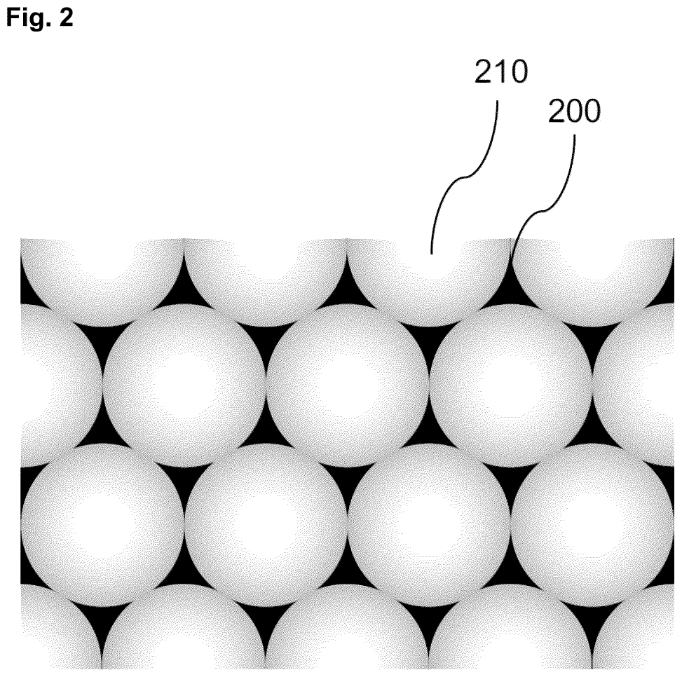

FIG. 2 depicts a 2-dimensional representation of a bed of close-packed microspheres encountered by cells and exogeneous agents when they are propelled into and through the bed of microspheres under the influence of centrifugal force. The triangular voids 200 found between the microspheres 210 provide sites for cell constriction to effect mechanoporation by cell squeezing. In 3-dimensional space, the microspheres are organized into regions having one of either hexagonal close-packed (HCP) and cubic close-packed (CCP) lattices, wherein the volume of the microspheres occupies from about 64% to about 74% of the total bed volume. In certain embodiments, 74% is the highest possible sphere density achievable.

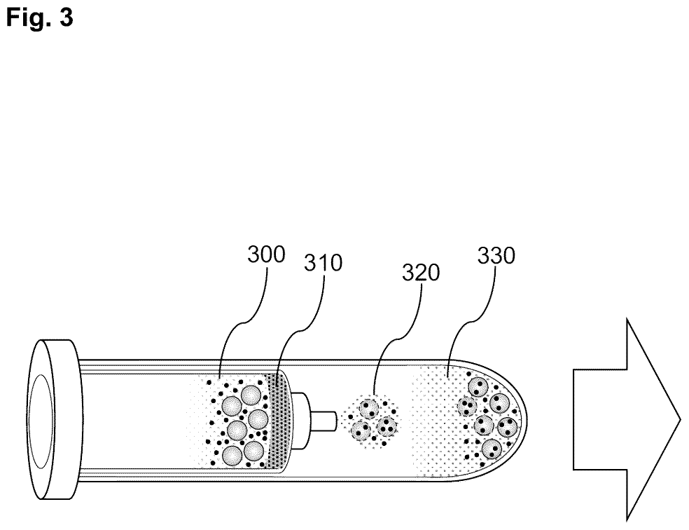

FIG. 3 depicts the processing of a spin column in a centrifuge where the arrow indicates the direction of centrifugal force. The spin column placed within the centrifuge tube is oriented as if being processed in a centrifuge fitted with a swinging bucket rotor. Cells and exogeneous cargo in the unprocessed volume 300 are propelled by centrifugal force through the bed of microspheres 310 where they are deformed by squeezing as they encounter triangular voids, and their porosity is temporarily altered enabling the cells to take up exogeneous agents. As cells emerge from the outlet of the spin column with their porosity altered 320, the cells continue to take up exogeneous cargo for a period of time from about 1 minute to about 30 minutes while their porosity is returning to normal. The unprocessed volume 300 must be sufficient to enable the cells to transverse the bed of microspheres before the volume of solution above the bed of microspheres is depleted. The centrifugal force both propels the cells through the bed of microspheres which facilitates the cell squeezing process and ensures the integrity of the bed of microspheres which is essential to the process.

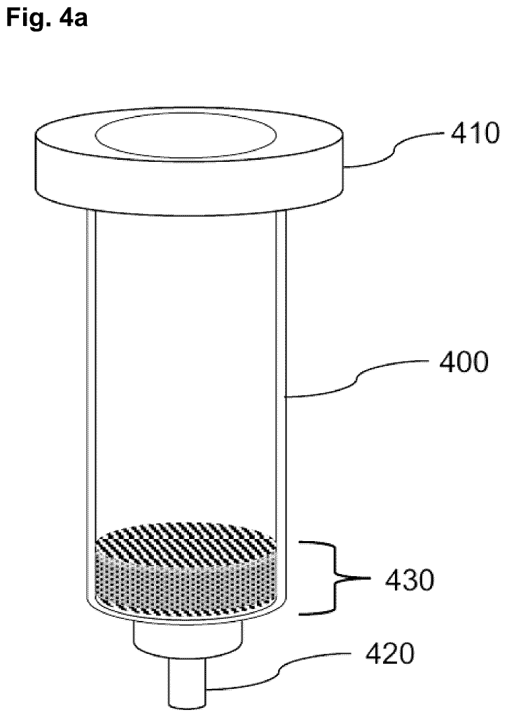

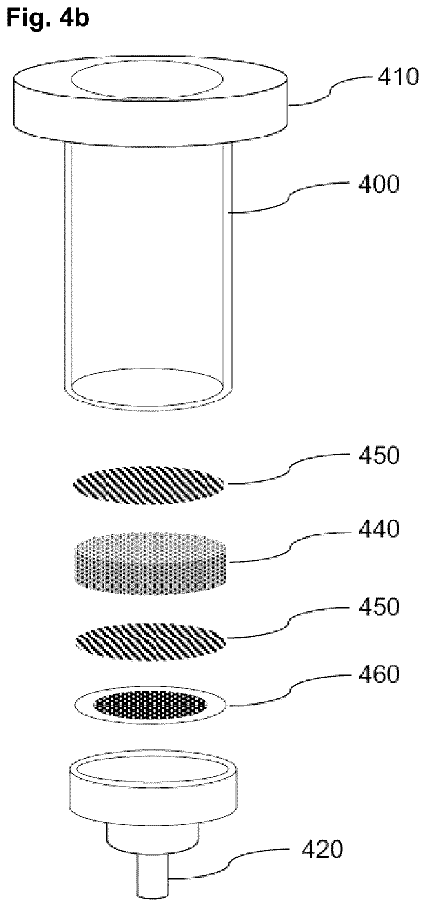

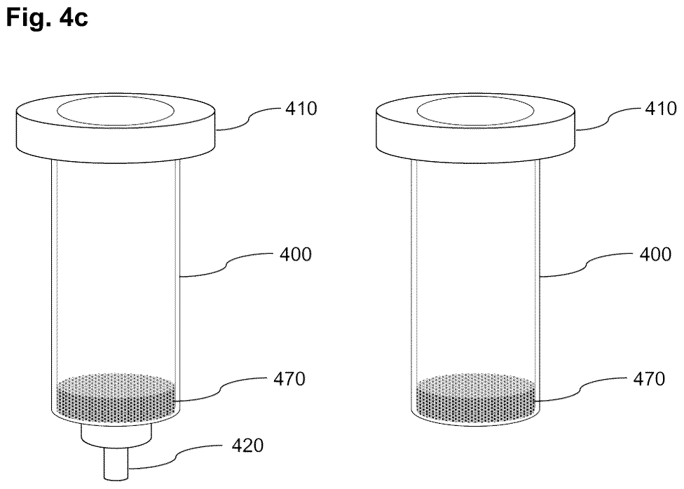

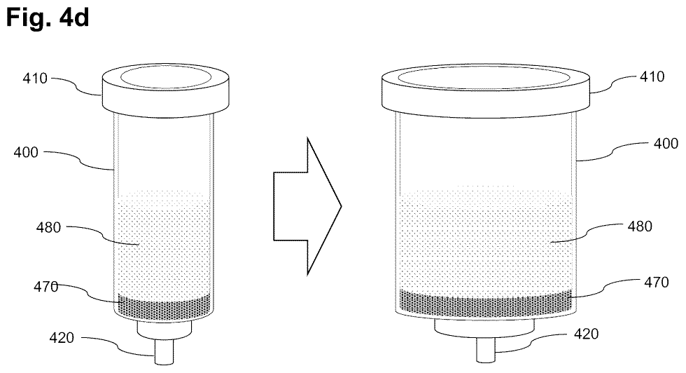

A device for microsphere facilitated intracellular delivery is depicted in FIGS. 4a through 4d in accordance with one or more embodiments of the present disclosure. FIG. 4a depicts a device comprised of a cylindrical column having an inlet and an outlet, in which the bed of microspheres resides above the outlet. The cylindrical column 400 has a flange at the inlet (top) 410 to support the column above the bottom of a centrifuge tube and a bed of microspheres with supporting structures 430 above the outlet (bottom) 420 which is depicted as a male luer adapter. The internal diameter of the column 400 is preferably from about 5 mm to about 25 mm, and the height of the bed of microspheres is preferably from about 0.2 mm to about 2.0 mm.

In certain embodiments, the bed of microspheres may be comprised of rigid, nonporous microspheres having a diameter of from about 3 μm to about 70 μm. In certain embodiments, the bed of rigid, nonporous microspheres have ranges of diameters, wherein the ranges are preferably selected from, but not limited to, one of from about 3 μm to about 10 μm, from about 20 μm to about 27 μm, from about 27 μm to about 32 μm, of from about 27 μm to about 45 μm, of from about 32 μm to about 38 μm, and of from about 38 μm to about 45 μm.

In certain embodiments, the microspheres may be organized into regions having one of either hexagonal close-packed, cubic close-packed and a combination of hexagonal and cubic close packed structures. In certain embodiments, the volume of the microspheres may occupy from about 64% to about 74% of the total bed volume.

By way of example, microspheres suitable for use in the method and device disclosed herein can be obtained commercially or prepared from one of metals, silica, alumina, titania, zirconia, glass, ceramics and organic polymers including, but not limited to, one of glycidyl methacrylate, polycaprolactone, polyetheretherketone, high density polyethylene, poly(methyl methacrylate), poly(lactic-co-glycolic acid), poly(L-lactic-co-hydroxymethyl glycolic acid) and polystyrene.

FIG. 4b depicts an exploded view of one embodiment of a device of the present disclosure in which the bed of microspheres in the cylindrical column 440 is supported by either a polymeric frit or membrane filter 450 placed either below the bed or both above and below the bed. The polymeric frit or membrane filter may retain the microspheres but permit the unrestricted passage of cells and exogeneous agents. The polymeric frit or membrane filter can be further supported by a metallic screen or porous polymeric disc 460. By way of example, suitable polymeric frits can be purchased or prepared from one of polyamide, polyethylene, high density polyethylene, polytetrafluoroethylene, polyvinylidene difluoride and Nylon 6, and suitable membrane filters can be purchased or prepared from polytetrafluoroethylene, polyvinylidene difluoride, polypropylene, polyethersulfone and polycarbonate.

FIG. 4c depicts two versions of a device of the present disclosure in which the bed of microspheres 470 is comprised of sintered polymeric microspheres prepared by sintering a compressed bed of microspheres at elevated temperature to produce a rigid bed of fused microspheres. In certain embodiments, the rigid bed of fused microspheres may be comprised of polymeric microspheres preferably selected from, but not limited to, one of either high density polyethylene (HDPE), polyetheretherketone (PEEK), poly(methyl methacrylate) (PMMA) and polystyrene (PS). In the “spin column” configuration (FIG. 4c, left), the outlet 420 is depicted as a male luer adapter. In the “spin cup” configuration (FIG. 4c, right), the outlet has no connector associated with it.

In certain embodiments, the bed of microspheres may be comprised of polymeric microspheres having a diameter of from about 3 μm to about 70 μm. In certain embodiments, the microspheres may have a narrow size distribution or monodispersity of from about 1% to about 10%. In certain embodiments, the microspheres may be organized into regions having one of either hexagonal close-packed, cubic close-packed and a combination of hexagonal and cubic close packed structures. In certain embodiments, the volume of the microspheres may occupy from about 64% to about 74% of the total bed volume.

FIG. 4d depicts two versions of a device of the present disclosure in which the bed of microspheres 470 is comprised of sintered polymeric microspheres prepared by sintering a compressed bed of microspheres at elevated temperature to produce a rigid bed of fused microspheres. In certain embodiments, the bed may be comprised of polymeric microspheres having a diameter of from about 3 μm to about 70 μm. In certain embodiments, the microspheres have ranges of diameters, wherein the ranges are preferably selected from, but not limited to, one of from about 3 μm to about 10 μm, from about 20 μm to about 27 μm, from about 27 μm to about 32 μm, of from about 27 μm to about 45 μm, of from about 32 μm to about 38 μm, and of from about 38 μm to about 45 μm.

In certain embodiments, the microspheres may be organized into regions having one of hexagonal close-packed and cubic close-packed lattices. In certain embodiments, the volume of the microspheres may occupy from about 64% to about 74% of the total bed volume. In certain embodiments, the internal diameter of the column may be preferably from about 5 mm to about 25 mm. In certain embodiments, the height of the bed of microspheres may be preferably from about 0.2 mm to about 2.0 mm. In certain embodiments, the height of the bed of microspheres may be preferably from about 0.5 mm to about 2.0 mm. The two versions of the device shown in FIG. 4d differ only with respect to their diameter. Nevertheless, they both require that the height of the solution containing the cells and exogenous agents be sufficient to enable the cells to transverse the bed of microspheres before the volume of the solution above the bed of microspheres is depleted.

In some embodiments, the microspheres reside within a cylindrical column having tubing connectors at an inlet and an outlet. In certain embodiments, the internal diameter of the column may be from about 5 mm to about 10 mm. In certain embodiments, the height of the bed of microspheres may be preferably from about 0.2 mm to about 2.0 mm. In certain embodiments, the height of the bed of microspheres may be from about 0.5 mm to about 2.0 mm. In certain embodiments, a plurality of cells in the presence of exogenous cargos may be either propelled through the bed of microspheres by a liquid pump or by external pressure applied to a liquid reservoir or pulled through the bed of microspheres by applying a vacuum to the outlet of the column.

The methods of microsphere facilitated intracellular delivery disclosed herein may be easily scaled-up by one or more of: 1) increasing the diameter of the device; 2) processing multiple columns (e.g., from 2 to 8 columns) simultaneously in the centrifuge depending upon the capacity of the centrifuge rotor; and 3) processing a spin column plate comprised of parallel columns (e.g., 12, 24, 48 or 96 columns) in a 2×3 format in a centrifuge rotor with a microwell plate adapter. With respect to the methods disclosed herein, the height of the solution containing the cells and exogenous cargo must remain constant with respect to the height of the bed of microspheres, and the height of the solution must be sufficient to enable the cells to transverse the bed of microspheres before the solution is depleted.

Although in some embodiments, the microspheres mentioned herein are suitable for use with the methods and devices disclosed herein without surface modification, in some embodiments, microspheres will preferably require surface modification to minimize their potential for ionic and nonspecific adsorption of cells and exogeneous cargos. Methods for surface modification of the microspheres disclosed herein are well documented and familiar to those with skill in the art of biocompatible surface chemistries.

The method of microsphere facilitated intracellular delivery leverages the physical properties of equal close-packed spheres. In geometry, close packing of equal spheres is a dense arrangement of congruent spheres in an infinite, regular arrangement (or lattice) wherein the greatest fraction of space occupied by spheres is given by π/3\sqrt{2}≈0.74048 (e.g., 74%). The same packing density can be achieved by alternate stackings of the same close-packed planes of spheres. Many crystal structures are based on a close-packing of a single kind of atom, or close-packing of large ions with smaller ions filling the spaces between them. The two simple regular lattices that achieve the highest packing density are called Hexagonal Close-Packed (HCP) and Cubic Close-Packed (CCP) based upon their symmetry. These arrangements are very close to one another in energy, and it is difficult to predict which form will be preferred from first principals.

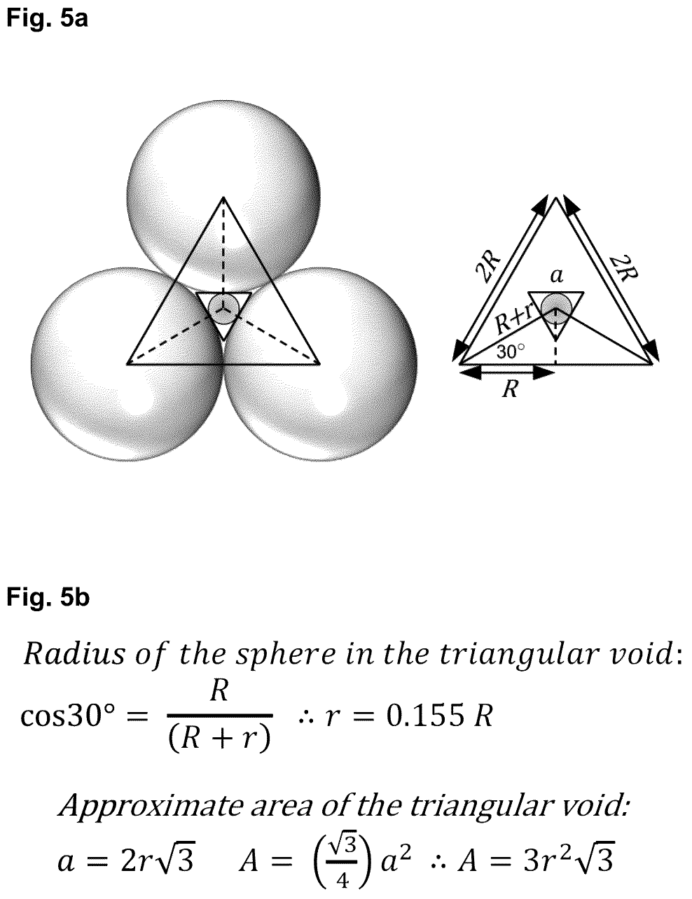

FIG. 5a illustrates the geometric relationships associated with the triangular voids found in planes of equal close-packed spheres, and FIG. 5b summarizes the derivation of equations to 1) determine the radius of a sphere inscribed within a triangular void, and 2) determine the approximate area of the triangular void. The radius of a sphere inscribed within a triangular void represents the maximum radius of a cell that can transverse the void without encountering a constriction. In studies involving crystal structures, the radius of a sphere inscribed within a triangular void represents the maximum radius of a counter ion that will fit within a crystal lattice. The area of the triangular void is approximated by the area of the equilateral triangle wherein the sphere of maximum radius is inscribed. Both the radius of the sphere in the rectangular void, and the approximate area of the triangular void can be derived from the radius of the spheres that make up the close-packed structure.

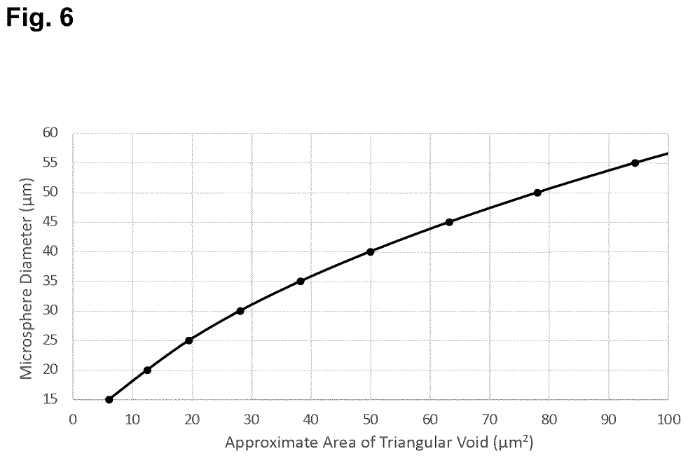

FIG. 6 shows a graphical representation of the relationship between the diameter of a microsphere and the approximate area of the corresponding triangular void. To explore the utility of triangular voids as constrictions for mechanoporation by cell squeezing, the cross-sectional area of the void must be compared to that of the cell to be processed. Table 1 below summarizes the dimensional properties of therapeutic and regenerative cells currently of interest with respect to cell squeezing.

TABLE 1
Representative dimensional properties of therapeutic and
regenerative cell lines. All values represent averages.

| | Cell | Cell | Cell |
|---|---|---|---|
| | Diameter | Area | Volume |
| Cell Type | (μm) | (μm²) | (μm³) |
| T-Cells and CAR T-cells | 8 | 50 | 268 |
| Natural Killer Cells (NK-Cells) | 9 | 64 | 382 |
| Neural Stem Cells (NSCs) | 15 | 177 | 1767 |
| Mesenchymal Stem Cells | 18 | 255 | 3054 |
| (MSCs) | | | |

FIG. 6 can be used to facilitate the selection of microspheres for mechanoporation by cell squeezing involving specific cell types. For example, to facilitate cell squeezing involving T-cells having an approximate diameter of 8 μm and an approximate cross-sectional area of 50 μm², the graph indicates that spheres with diameters of less than 40 μm afford triangular voids with areas of less than 50 μm² that may exhibit utility with respect to cell squeezing. The cross-sectional area of the constriction should ideally be from about 30% to about 80% of that of the target cell for efficient cell squeezing. The graph indicates that microspheres with diameters of from about 15 μm to about 20 μm should afford triangular voids with cross-sectional areas of from about 12% to about 20% of that of a T-cell, and microspheres with diameters of from about 25 μm to about 30 μm should afford triangular voids with cross-sectional areas of from about 39% to about 56% of that of a T-cell. Therefore, microspheres with diameters in range of from about 20 μm to about 30 μm should facilitate efficient cell squeezing of T-cells. This general approach may be employed to select microspheres of appropriate diameters to facilitate cell squeezing involving cells of any diameter. In certain embodiments, microspheres having the diameters that appear in FIG. 6 may be made of, for example, without limitation, glass, polymeric, or silica.

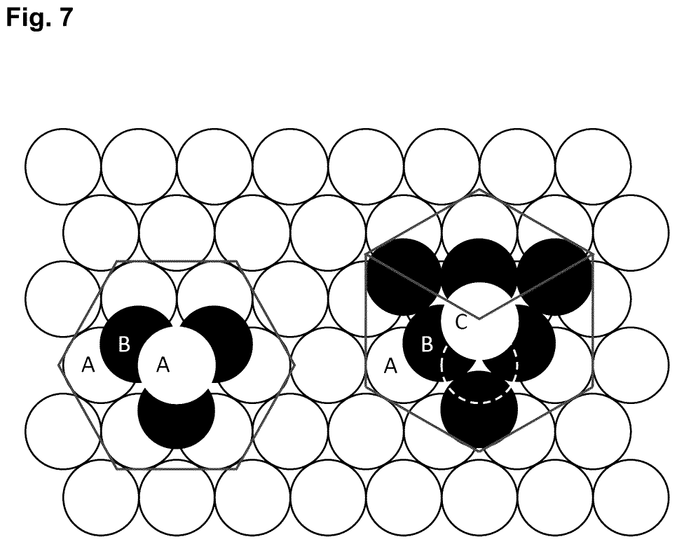

FIG. 7 depicts diagonal close-packed and cubic close-packed lattices involving equal spheres. In perfect lattices free of defects, 74% of the available space is occupied by spheres. Both HCP and CCP lattices have been well characterized with respect to their 3-dimensional arrangements. With reference to FIG. 7, the HCP lattice is depicted as being comprised of two alternating planes of equal spheres designated “A” and “B,” wherein all “A” planes and “B” planes are perfectly aligned, and the CCP lattice is depicted as being comprised of three alternating planes designated “A,” “B,” and “C” planes, wherein all “A,” “B” and “C” planes are perfectly aligned. Packing spheres in any pattern generates some empty space between them and these gaps are known as interstitial spaces or voids.

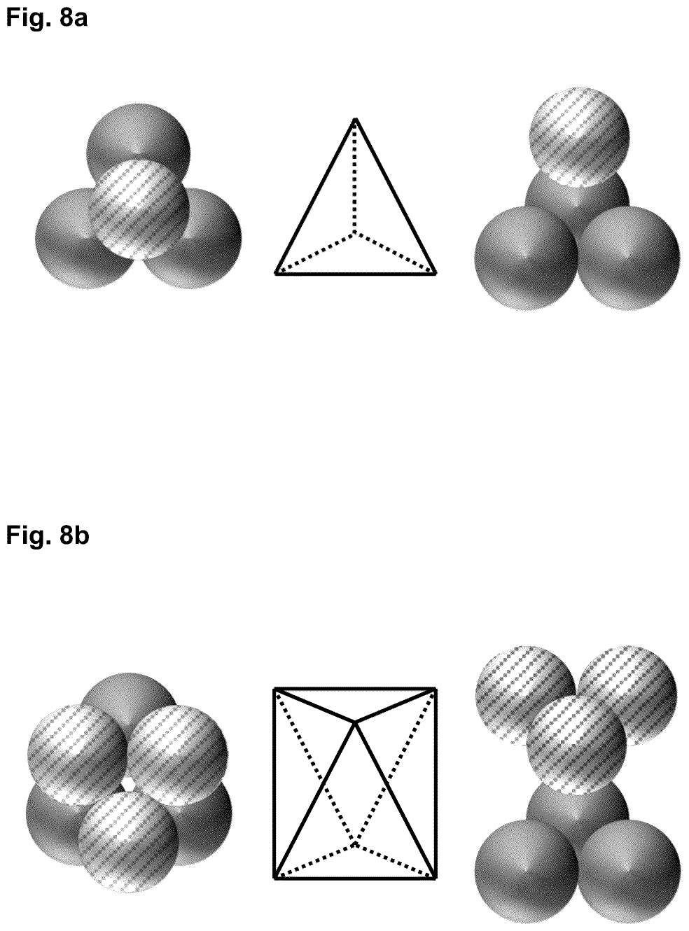

FIG. 8a uses space-filling and wireframe models to depict the 3-dimensional properties of the tetrahedral void associated with the HCP lattice, and FIG. 8b uses space-filling and wireframe models to depict the 3-dimensional properties of the octahedral void associated with the CCP lattice. There are twice as many tetrahedral voids as octahedral voids in close-packed lattices.

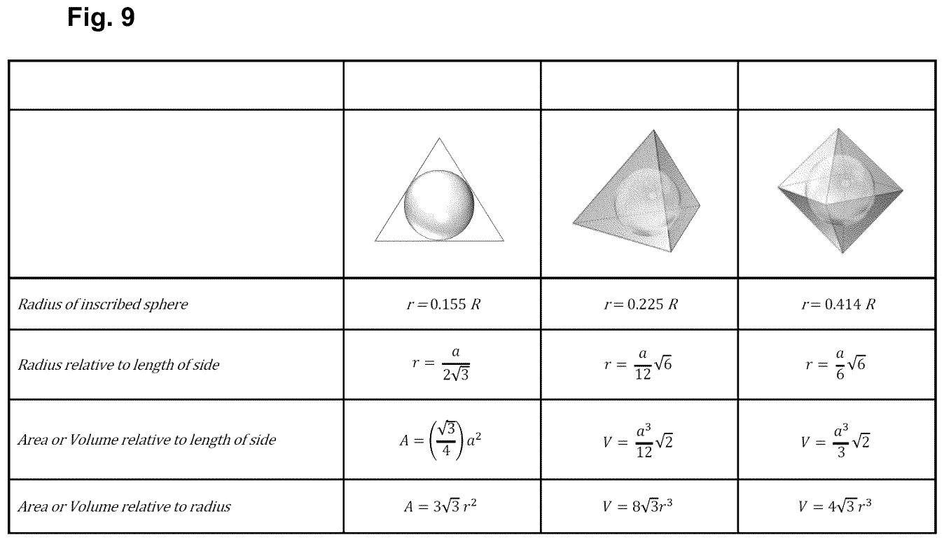

To understand the impact that tetrahedral and octahedral voids may have upon mechanoporation by cell squeezing it is necessary to estimate the volumes of the voids as a function of microsphere radius and compare these estimates to the volumes of the cells to be processed. FIG. 9 summarizes the equations used to estimate the volumes of the tetrahedral and octahedral voids as a function of the radii of spheres inscribed within the voids. The radii of the inscribed spheres for each of the voids are derived by trigonometry, and these radii include: 0.155, 0.225 and 0.414 for the triangular void, tetrahedral void and octahedral void, respectively.

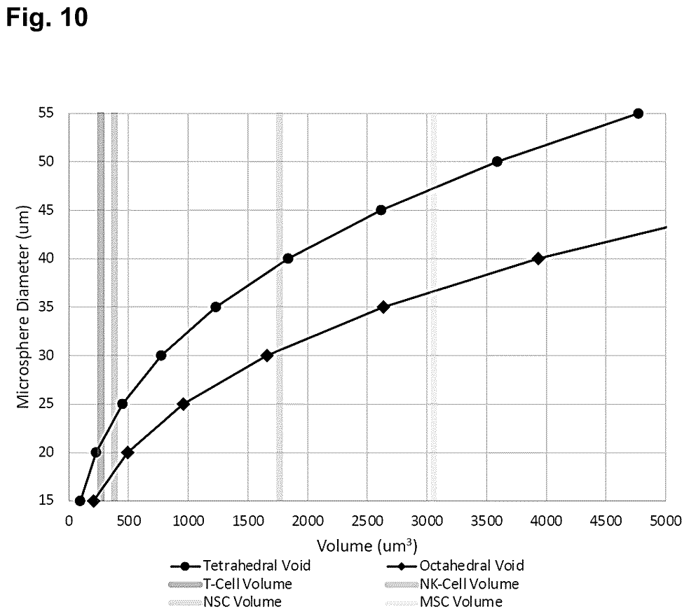

FIG. 10 shows a graphical representation of the relationship between the diameter of a microsphere and the approximate volume of the corresponding tetrahedral and octahedral voids. FIG. 10 also shows the approximate volumes of therapeutic and regenerative cells currently of interest with respect to cell squeezing (from Table 1). Considering the example involving the processing of T-cells presented herein, it was reasoned that microspheres with diameters of from about 20 μm to about 30 μm should afford triangular voids with cross-sectional areas of from about 20% to about 56% of that of a T-cell, and that microspheres with diameters in this range should exhibit utility with respect to mechanoporation by cell squeezing. FIG. 10 shows that microspheres with diameters of from about 20 μm to about 30 μm afford tetrahedral voids with volumes approximately equal to or greater than that of a T-cell. Consequently, it can be assumed that for microspheres with diameters in the range of from about 25 μm to about 30 μm sufficient space is available within the tetrahedral voids for T-cells to fully recover their initial volume before encountering the next triangular void (constriction). For several cell squeezing events to happen, cell recovery in those voids may be necessary in between the transitions from cell constriction void to another cell constriction void. Consequently, FIG. 10 can be used to narrow the selection of microspheres to those that afford voids that will allow the cells to fully recover their initial volumes before encountering further constrictions. Note that the actual extent to which cells recover their initial volumes is also a function of centrifuge speed.

Note that for a specific microsphere diameter the volume of the corresponding octahedral void is approximately twice that of the corresponding tetrahedral void. It is important to appreciate that for a specific microsphere diameter the calculated volumes of the tetrahedral and octahedral voids represent significant underestimates in that additional volume is available for cells to occupy beyond the limits of the polyhedral approximations because the faces of the polyhedrons contact the surrounding spheres at a single point of contact with adjacent spheres and the volume associated with the receding surfaces of the adjacent spheres is not considered.

As discussed herein, the preferred height of the bed of microspheres is about 0.2 mm to about 2.0 mm. The height of the packed bed determines how many times the cell squeezing process is repeated as cells travel through the bed of microspheres. Assuming cells transverse one triangular void that acts as a constriction per plane of microspheres encountered, the number of squeezing events is estimated by dividing the height of the bed of microspheres by the diameter of the microspheres (e.g., a 1 mm bed height (1000 μm) prepared from 50 μm microspheres should afford 20 cell-squeezing events). This represents an overestimate because statistically 1 time out of every 3 when exiting a constriction, a cell will encounter an octahedral void that will enable the cell to skip one plane of microspheres before encountering the next triangular void that acts as a constriction. Once an optimum bed height is determined for a specific cell type in conjunction with specific exogeneous cargos, the process can be easily scaled-up by one or more of: 1) increasing the diameter of the device; 2) processing multiple columns (e.g., from 2 to 8 columns) simultaneously in the centrifuge depending upon the capacity of the centrifuge rotor; and 3) processing a spin column plate comprised of parallel columns (e.g., 12, 24, 48 and 96) in a 2×3 format in a centrifuge rotor with a microwell plate adapter.

### EXAMPLES

The foregoing and the following examples are merely intended to illustrate various embodiments of the present disclosure. The specific modifications discussed above are not to be construed as limitations on the scope of the disclosure. It will be apparent to one skilled in the art that various equivalents, changes, and modifications may be made without departing from the scope of the disclosure, and it is understood that such equivalent embodiments are to be included herein.

### Example 1. Microsphere Facilitated Delivery of Nanoparticles to T-Cells

As described in more detail below, peripheral blood-derived T-cells were modified by incorporation of VMI-Trac Ultra nanoparticles, which are iron-containing protein nanoparticles comprised of heparin, protamine and ferumoxytol with an average diameter of 140 nm. In the presence of the nanoparticles, T-cells were driven through polyetheretherketone (PEEK) frits made of microparticles to facilitate intracellular delivery of the nanoparticles to the T-cells.

First, three different PEEK frits having average porosities of 2 μm, 5 μm, or 10 μm were prepared by sintering PEEK microspheres at an elevated temperature. The 2 μm porosity PEEK frit had an approximate microsphere diameter of 13 μm, the 5 μm porosity PEEK frit had an approximate microsphere diameter of 32 μm, and the 10 μm porosity PEEK frit had an approximate microsphere diameter of 64 μm. PEEK frits were fitted in spin columns with internal diameters of 6.8 mm. The height of the PEEK frits was 1.8 mm. The volume of the 2 μm porosity PEEK frit was 24% therefore the total volume (density) of the microspheres in the PEEK frit was 75%; the volume of the 5 μm porosity PEEK frit was 26% therefore the volume (density) of the microspheres was 74%; and the volume of the 10 μm porosity PEEK frit was 28%, therefore, the volume (density) of the microspheres was 72%. The spin columns were placed in centrifuge tubes and washed and equilibrated by adding 750 μl of serum free media to the columns above the frits, followed by centrifugation in a swinging-bucket rotor, and discarding of flow through.

Next, peripheral blood-derived T-cells were collected and pelleted via centrifugation. The T-cell pellet was resuspended with a labeling mixture containing VMI-Trac Ultra nanoparticles, VMI-Trac Duo reagent (containing a contrast agent used to fluoresce the VMI-Trac Ultra nanoparticles), and serum free media, and the resulting solution was transferred to the spin columns containing the PEEK frits. The spin columns were placed in centrifuge tubes in a swinging-bucket rotor and then centrifuged for 3 minutes at 1500 rpm (˜483×g) to propel the solution containing the cells in the presence of the VMI-Trac nanoparticles and reagent through the PEEK frits. After centrifugation, the processed cells were transferred from the centrifuge tube to the well of a 24-well plate. A volume of complete media containing 20% fetal bovine serum (FBS) was added to the cells (resulting in 10% FBS final concentration) and the cells were allowed to recover in a 37° C. tissue culture incubator for 120 minutes. Following cell recovery (incubation), the cells were collected, and the presence of VMI-Trac Ultra nanoparticles was determined by Prussian Blue staining and fluorescence. Cell viability and recovery were determined by trypan blue staining.

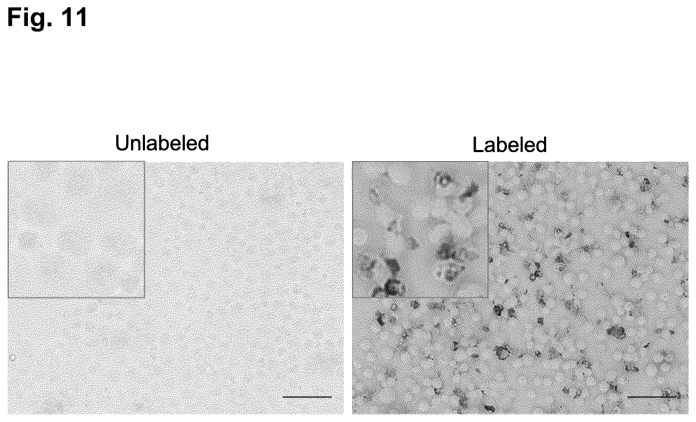

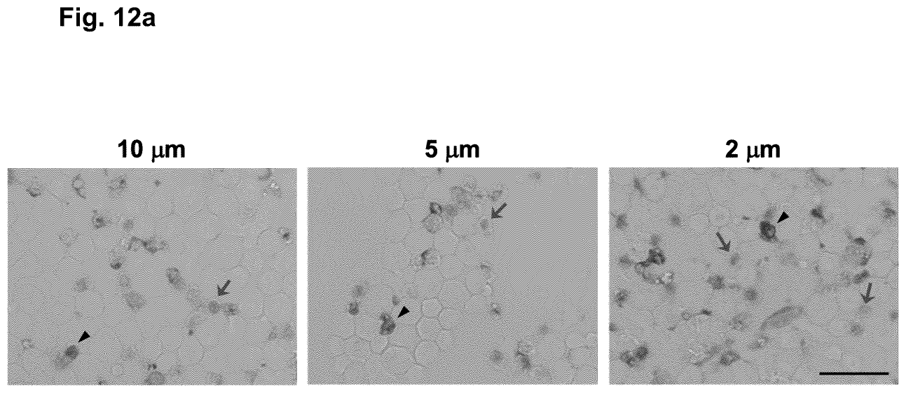

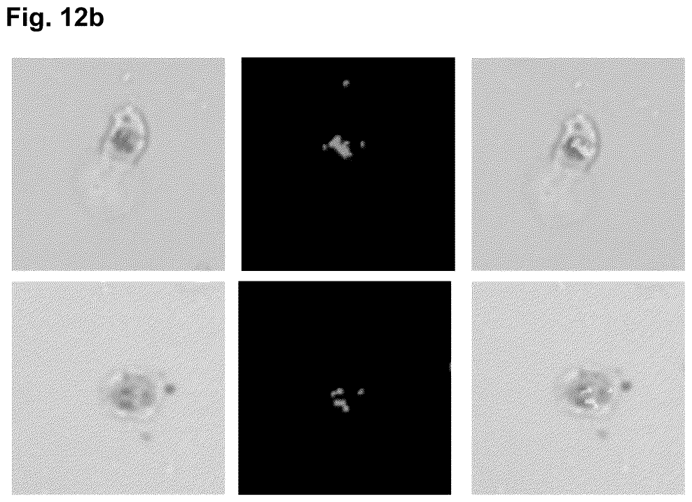

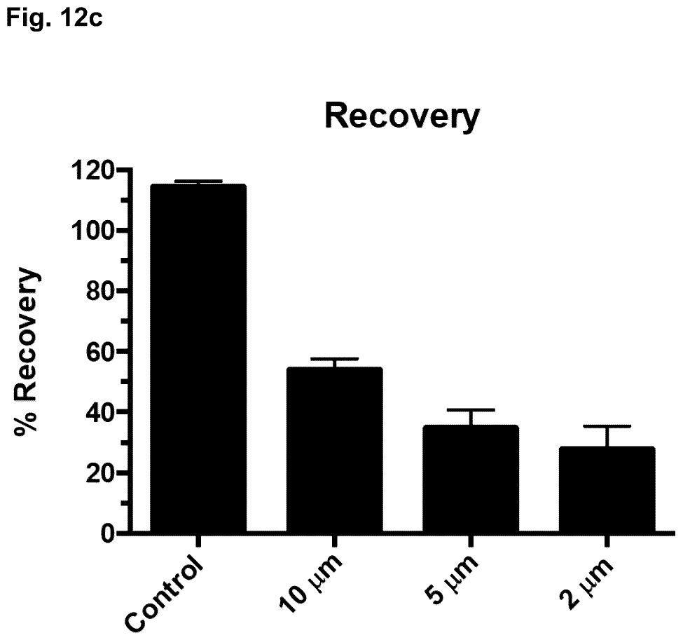

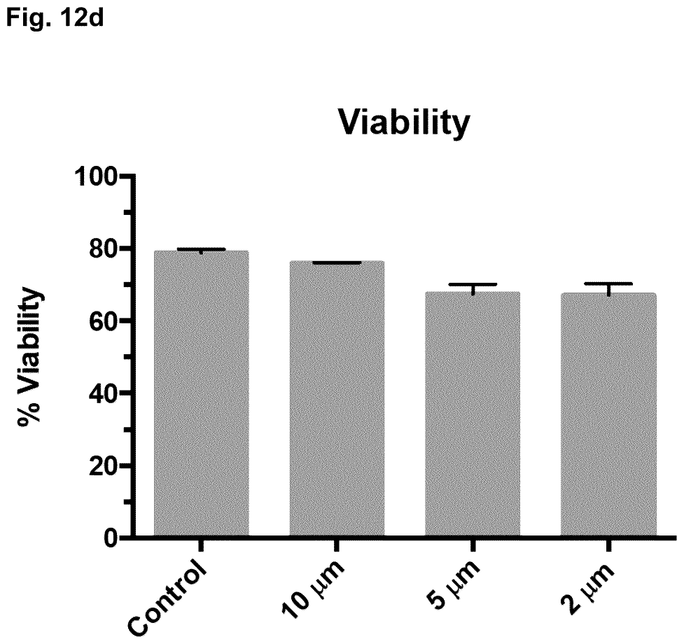

Prussian Blue staining was performed to detect iron-positive VMI-Trac Ultra labeled (incorporated) cells. Cells were pelleted and then washed twice with Dulbecco's phosphate-buffered saline (DPBS). For cell fixation, 4% paraformaldehyde was added to the washed cells, which were incubated at room temperature for 30 minutes. Fixed cells were washed with distilled H₂O and spotted onto a gelatin coated glass slide. Finally, freshly prepared Prussian Blue staining solution (mix equal parts 5% Potassium Ferrocyanide and 5% hydrochloric acid) was then added to the fixed cells which were incubated at room temperature for 1 hour. The resulting microscope images of cells stained with Prussian Blue to detect the presence of iron (i.e., the iron based VMI-Trac Ultra nanoparticles) are shown in FIG. 11 (right panel, “Labeled” showing results from PEEK frit with an average porosity of 10 μm), FIG. 12a (showing results from PEEK frit with average porosities of 10 μm, 5 μm, or 2 μm), and 12b (left panels, showing results from PEEK frit with an average porosity of 10 μm). As shown in FIGS. 11, 12a and 12b (left panels), VMI-Trac Ultra nanoparticles were successfully incorporated into T-cells via microsphere facilitated delivery using PEEK frits with average porosities of 10 μm, 5 μm, or 2 μm (and microsphere diameters of 64 μm, 32 μm, or 13 μm, respectively). Next, fluorescence was used to visualize the presence of VMI-Trac Ultra nanoparticles labeled with the fluorescent VMI-Trac Duo reagent (FIG. 12b, middle panels). FIG. 12b (right panels) is an alignment of the images of cells stained in Prussian Blue (FIG. 12b, left panels) and cells visualized with fluorescence (FIG. 12b, middle panels), which confirms by both Prussian Blue staining for iron-positive cells and fluorescence detection that the VMI-Trac Ultra nanoparticles were internalized into the T-cells and were not adhering to the cell surface. The total cell recovery and viability of T-cells containing VMI-Trac Ultra nanoparticles is shown in FIG. 12c and FIG. 12d. In sum, the results demonstrate successful microsphere facilitated intracellular delivery of nanoparticles into T-cells. Moreover, the results indicate that the smaller the porosity of the frit, the greater the efficiency of intracellular delivery. This is consistent with the approach wherein the 2 μm frit squeezes the T-cell to about 20% of the cell's diameter which should provide efficient delivery, whereas the 5 μm frit only squeezes the T-cell to about 50% of the cell's diameter, which is less efficient, and the 10 μm frit squeezes the T-cell to a lesser extent.

### Example 2. Preparation of Sintered Microsphere Frits and Spin Columns with Sintered Microsphere Frits

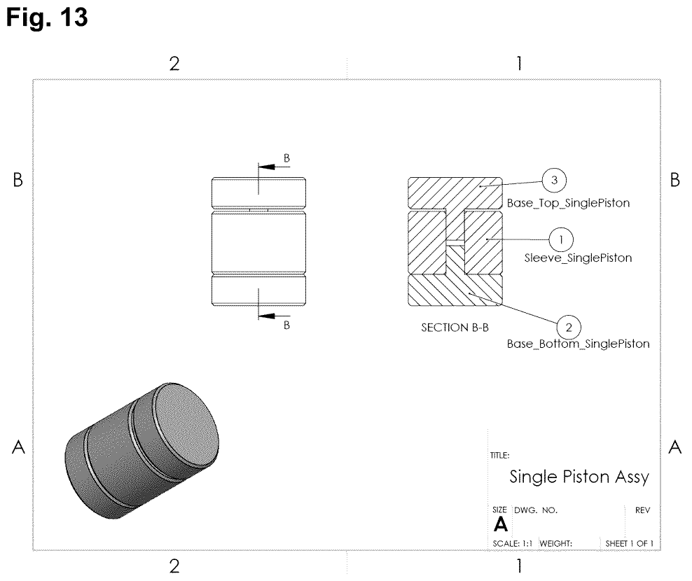

Cylindrical steel dies having an internal diameter of 7.4 mm, an external diameter of 25 mm, and a height of 15 mm were machined, and fitted with removable cylindrical upper and lower pistons having external diameters of slightly less than 7.4 mm and heights of 25 mm (FIG. 13). The upper pistons were removed and 400 mg of either 10 μm, 20 μm, or 30 μm diameter poly(methyl methacrylate) microspheres (Lab 261) was placed in the wells of individual dies (FIG. 13). The upper pistons were inserted, and the assemblies were placed adjacent to one another between ½″ thick steel plates in a 10-ton bench top press. The assembly was compressed at 7.5 tons for 15 min. The bodies of the dies were carefully removed, and the compressed cakes were transferred from the tops of the lower pistons to a ceramic boat with a small spatula. The ceramic boat was placed in an oven and the compressed cakes were sintered at 150 degrees C. for 24 hours. After the oven cooled to room temperature, the sintered frits were removed from the oven and inspected for uniformity by light microscopy at 40× magnification. If the frits exhibited sufficient uniformity (i.e., no evidence of significant voids), they were interference fit into the bodies of spin columns having 7.4 mm internal diameters (Biocomma 7400). The sintered microsphere frits were positioned immediately above hydrophilic 20 μm porosity PMMA frits that were used as supports due to the fragility of the sintered microsphere frits with heights of approximately 0.2 mm. The sintered microsphere frits were secured from above with locking rings with 1 mm flanges to ensure that liquid would not flow around the outer diameter of the frit.

Next, peripheral blood-derived T-cells were collected and pelleted via centrifugation. The T-cell pellet was resuspended with a labeling mixture containing VMI-Trac Ultra nanoparticles, VMI-Trac Duo reagent (containing a contrast agent used to fluoresce the VMI-Trac Ultra nanoparticles), and serum-free media, and the resulting solution was transferred to the spin columns containing the sintered microsphere frits. The spin columns were placed in centrifuge tubes in a swinging-bucket rotor and then centrifuged for 3 minutes at 1500 rpm (˜483×g) to propel the solution containing the cells in the presence of the VMI-Trac nanoparticles and reagent through the sintered microsphere frits. After centrifugation, the processed cells were transferred from the centrifuge tube to the well of a 24-well plate. A volume of complete media containing 20% fetal bovine serum (FBS) was added to the cells (resulting in 10% FBS final concentration) and the cells were allowed to recover in a 37° C. tissue culture incubator for 120 minutes. Following cell recovery (incubation), the cells were collected, and the presence of VMI-Trac Ultra nanoparticles was determined by Prussian Blue staining and fluorescence. Cell viability and recovery were determined by trypan blue staining.

### Example 3. Preparation of Packed Beds of Microspheres in Spin Columns

The blunt end of a 7.4 mm drill bit blank was used as a template to cut 7.4 mm diameter 10-micron nylon net filters (Millipore 1009000) and rigid plastic mesh (McMaster-Carr) with an X-acto knife. Rigid plastic mesh supports followed by filters were placed into the bodies of spin columns (Biocomma 7400) and were secured from above with locking rings with 1 mm flanges (reducing the available surface area to a 5.4 mm diameter circle) to ensure that liquid will not flow around the outer diameter of the filter. 400 mg of either 20 μm, 25 μm or 30 μm diameter poly(methyl methacrylate) microspheres (Lab 261) were placed into the spin columns followed by 500 μL of deionized water, and the microspheres were suspended in deionized water by vortexing. Packed beds of microspheres were obtained by centrifugation in a swinging-bucket rotor for 3 minutes at 3000 rpm. Prior to use, the spin columns were fitted with additional nylon net filters with rigid plastic mesh supports above the packed beds to maintain the integrity of the packed beds and ensure that the addition of cells and exogenous cargo will not disturb the surface of the packed beds prior to centrifugation. The packed beds of microspheres were conditioned (wetted) prior to use by centrifugal washing with 3 volumes of cell culture media.

Next, peripheral blood-derived T-cells were collected and pelleted via centrifugation. The T-cell pellet was resuspended with a labeling mixture containing VMI-Trac Ultra nanoparticles, VMI-Trac Duo reagent (containing a contrast agent used to fluoresce the VMI-Trac Ultra nanoparticles), and serum-free media, and the resulting solution was transferred to the spin columns containing packed beds of microspheres. The spin columns were placed in centrifuge tubes in a swinging-bucket rotor and then centrifuged for 3 minutes at 1500 rpm to propel the solution containing the cells in the presence of the VMI-Trac nanoparticles and reagent through the packed beds. After centrifugation, the processed cells were transferred from the centrifuge tube to the well of a 24-well plate. A volume of complete media containing 20% fetal bovine serum (FBS) was added to the cells (resulting in 10% FBS final concentration) and the cells were allowed to recover in a 37° C. tissue culture incubator for 120 minutes. Following cell recovery (incubation), the cells were collected, and the presence of VMI-Trac Ultra nanoparticles was determined by Prussian Blue staining and fluorescence. Cell viability and recovery were determined by trypan blue staining.

### Example 4: Delivery of CRISPR-Cas Ribonucleic Acid Protein to Human CD34+Hematopoietic Stem and Progenitor Cells

The CRISPR-Cas system holds tremendous potential not only for basic research but for therapeutic applications for knocking out or repairing genes. There are currently several approaches of delivering RNA-guided nucleases (e.g., Cas9) into cells of interest which include, viral delivery of the RNA-guided nucleases and guide RNAs, plasmid and mRNA delivery via lipid nanoparticles, or electroporation. These methods pose the risk of random integration of the RNA-guided nucleases/gRNA sequences into the genome, which can lead to undesired consequences. Theoretically, only a single protein reaching the target genomic sequence is needed, but these methods can generate a high expression of RNA-guided nucleases and gRNAs within the cell, which can tax the cell's protein synthesis mechanism with RNA-guided nucleases or even increase the risk of off target and random cutting. In addition, plasmid DNA and mRNA have proven to be quite bulky and thus difficult for transient delivery into suspension cells like human hematopoietic stem and progenitor cells (HSPCs) and T-cells. Due to the limited intrinsic endocytosis activity, HSPCs require a physical direct delivery method like electroporation. However, electroporation can be quite harsh to cells, leading to high cytotoxicity and reduced proliferation.

In this example, the delivery of ribonucleoprotein (RNP) complexes (RNA-guided nuclease complexed with a gRNA) is described. In certain embodiments, the cargo is smaller, has a shorter lifespan, and poses no risk of random integration. In certain embodiments, any RNA-guided nuclease (e.g., Cas protein (e.g., Cas9)) may be used in combination with gRNAs for delivery to cells. In this example, microsphere facilitated intracellular delivery using the device depicted in FIGS. 4a through 4d may be performed, which enables the delivery of RNP complexes. For example, a sintered microsphere frit may be prepared from 30 μm diameter poly(methyl methacrylate) microspheres as described in Example 2 above. A Cas9 protein may be obtained commercially and formulated in 20 mM HEPES, 150 mM KCl, 1% Sucrose, 1 mM TCEP at pH 7.5 or 20 mM TRIS-HCl at pH 8.0, 200 mM KCl, 10 mM MgCl₂. In certain embodiments, the gRNA molecules may be synthesized. In certain embodiments, the gRNA molecule may include a targeting domain that is complementary to a sequence in a target genome. In certain embodiments, the gRNA may include a targeting domain complementary to a sequence of beta-2-microglobulin (B2M). In certain embodiments, the B2M targeting domain sequence may be GGCCGAGAUGUCUCGCUCCG (SEQ ID NO: 1). Frozen bone marrow CD34+HSPCs may be purchased from Lonza. StemSpan SFEM II media (09655), CC100 (02690), may be purchased from Stem Cell Technologies. The spin column containing the microsphere frit may be conditioned by centrifuging 3 column volumes of 20 mM TRIS-HCl at pH 8.0, 200 mM KC!, 10 mM MgCl₂ prior to use.

Frozen bone marrow-derived CD34+HSPCs may be purchased from Lonza or AllCells and thawed according to manufacturer's instruction. Cells may be cultured in SFEM II supplemented with CCII0 (StemCell Technologies), 0.75 μM StemRegenin 1 (StemCell Technologies), 50 nM UMI 71 (StemCell Technologies), 50 ng/mL human recombinant IL-6 (Peprotech), and PenStrep. Cells may be cultured/expanded for 3-5 days before being used in experiments.

Human hematopoietic stem and progenitor cells (6 million cells) may be spun down and resuspended in 20 μL of SFEMII media. 200 μg of Cas9 may be mixed with 40 μg of gRNA targeting B2M and allowed to complex by incubating for 10 minutes at room temperature. The RNP mixture may be mixed with the cells and allowed to incubate for 2 min at room temperature with a final volume of 50 μL. The mixture may then be transferred into a spin column, placed in a 2 mL centrifuge tube and transferred to a swinging-bucket rotor in a microfuge. A second spin column also containing 50 μL of SFEMII media may be used as a counter-balance. The spin columns may be centrifuged for 3 minutes at 1500 rpm (˜483×g) to propel the solution containing the CD34+HSPCs in the presence of Cas9 and gRNA targeting B2M through the sintered microsphere frit.

The flow-through may be allowed to rest for from 2-5 minutes, before the sintered microsphere frit may be washed with 0.5 mL of complete media and the flow-through fractions combined. After rest, the cells may then be supplemented with fresh SFEM II media supplemented with StemRegenin 4-[2-[[2-(I-benzothiophen-3-yl)-9-propan-2-ylpurin-6-y 1]amino]ethyl]phenol) at 1 million cells/mL. The cells may be allowed to recover and expand ex vivo for 72 hours before analysis. Finally, the cells may be analyzed with respect to recovery, editing efficiency, and antibody staining to detect B2M knockdown.

The above-detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method for intracellular delivery comprising:
(a) passing a solution comprising a plurality of cells and one or more exogeneous cargo through a matrix comprising a plurality of microspheres wherein the one or more exogenous cargo is delivered to the plurality of cells, and
(b) incubating the solution for a period of time after passing the solution through the matrix.

2. The method of claim 1, wherein the microspheres have a diameter of from about 3 μm to about 70 μm.

3. The method of claim 2, wherein the microspheres have a monodispersity of from about 1% to about 10%.

4-8. (canceled)

9. The method of claim 3, wherein the microspheres comprise one or more materials selected from the group consisting of metal, silica, alumina, titania, zirconia, glass, ceramic, and organic polymer.

10-12. (canceled)

13. The method of claim 9, wherein passing the solution occurs via centrifugal force or pressure selected from the group consisting of pressure from a liquid pump, pressure from a liquid driven reservoir, pressure from a high-pressure gas source, external pressure applied to a liquid reservoir, pressure from application of a vacuum to the outlet, and a combination thereof.

14-16. (canceled)

17. The method of claim 13, wherein the one or more exogenous cargo is selected from the group consisting of a therapeutic molecule, a gene editing tool, a reprogramming factor, a genetic modification tool, an intracellular sensor, and an intracellular device.

18-26. (canceled)

27. The method of claim 17, wherein
the microspheres are comprised of sintered microspheres.

28. (canceled)

29. A device for intracellular delivery comprising:
(a) a column,
(b) a matrix comprising a plurality of microspheres in the column.

30. The device of claim 29, wherein the column has an inlet and an outlet, and wherein the matrix is positioned above the outlet.

31. (canceled)

32. The device of claim 30, wherein the microspheres have a diameter of from about 3 μm to about 70 μm.

33. The device of claim 32, wherein the microspheres have a monodispersity of from about 1% to about 10%.

34. (canceled)

35. The device of claim 33, wherein the matrix comprises hexagonal close-packed lattices, cubic close-packed lattices, or hexagonal close-packed lattices and cubic close-packed lattices.

36-38. (canceled)

39. The device of claim 35, wherein the microspheres comprise one or more materials selected from the group consisting of metal, silica, alumina, titania, zirconia, glass, ceramic, and organic polymer.

40-46. (canceled)

47. A method for intracellular delivery comprising:
(a) preparing a total volume of a solution in a column, the solution comprising
a plurality of cells and
one or more exogeneous cargo,
the column comprising a matrix comprising a plurality of microspheres,
wherein the total volume of the solution comprises a volume of the solution above the matrix and a volume of solution within the matrix;
(b) passing the total volume of solution through the matrix,
wherein the one or more exogenous cargo is delivered to the plurality of cells, and
wherein the volume of the solution above the matrix allows the plurality of cells to pass through the matrix before the total volume of the solution passes through the matrix; and
(c) incubating the solution for a period of time after passing the solution through the matrix.

48. The method of claim 47, wherein the microspheres have a diameter of from about 3 μm to about 70 μm.

49. The method of claim 48, wherein the microspheres have a monodispersity of from about 1% to about 10%.

50-54. (canceled)

55. The method of claim 49, wherein the microspheres comprise one or more materials selected from the group consisting of metal, silica, alumina, titania, zirconia, glass, ceramic, and organic polymer.

56-58. (canceled)

59. The method of claim 55, wherein passing the solution occurs via centrifugal force or pressure selected from the group consisting of pressure from a liquid pump, pressure from a liquid driven reservoir, pressure from a high-pressure gas source, external pressure applied to a liquid reservoir, pressure from application of a vacuum to the outlet, and a combination thereof.

60-62. (canceled)

63. The method of claim 59, wherein the one or more exogenous cargo is selected from the group consisting of a therapeutic molecule, a gene editing tool, a reprogramming factor, a genetic modification tool, an intracellular sensor, and an intracellular device.

64-72. (canceled)

73. The method of claim 63, wherein the microspheres are comprised of sintered microspheres.

74. (canceled)
